(19) United States
(12) Patent Application Publication
(10) Pub. No.: US 2007/0178145 A1
(43) Pub. Date: Aug. 2, 2007

(54) Three dimensional slow release agent absorbent dressing

(76) Inventors: Chang-Tsung Chou, Taipei City (TW); Chia-Ju Shih, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/342,802

(22) Filed: Jan. 31, 2006

(51) Int. Cl. A61L 15/00

(52) U.S. Cl. 424/445; 442/123

(57) ABSTRACT

A three-dimensional slow release agent absorbent dressing particularly suitable for use in sinus cavity or infective wounds comprises a wound-contacting layer surrounding outside of the dressing. The wound-contacting layer is a soft-film covered with tapered apertures and the bottom surface of said tapered apertures faces outside to contact a wound for discharging exudate from the wound in many directions and transmitting exudate via a diffusion guiding layer to absorbent articles. Said absorbent articles formed of high-molecular polymeric fibers are mixed with a certain concentration of water-soluble agents, such as antiseptic agents, enzymes and growth factor agents, in a suitable amount. After exudate is into absorbent articles, said polymeric fibers expand forming into the shape of gel to avoid exudate flowing backward to the wound, which is more effective in preventing the wound from secondary infection.

### BACKGROUND OF THE INVENTION

1. Field of the Invention

The present invention relates to a three-dimensional slow release agent absorbent dressing more suitable for use in sinus cavity or infective wounds. A first outside layer of said dressing is a wound-contacting layer formed of a porous soft-film with tapered apertures, which have a function of absorbing exudate from a wound quickly to avoid exudate flowing backward to the wound. Moreover, a certain concentration of water-soluble agents, such as antiseptic agents, enzymes and growth factor agents, in a suitable amount is able to be mixed into dry absorbent articles of the dressing of the present invention, which is more effective in infection control, debridement and wound-healing.

2. Prior Art

Almost every healing process general adopted in the medical institution is using antimicrobial dressings to cover or bandage wounds, such as laceration, surgical incision, inflammation or burns for preventing them from being infected by external bacteria. However, the structure of dressings used commonly is a flat form and they are only fit to be applied on surface wounds. But some wounds are deep and even the depth of wounds can be five centimeters. If people still force to insert common dressings into such deep wounds, wounds might get more serious. In that case, it is definitely not convenient to use general dressings to heal deep wounds like sinus cavity wounds.

### OBJECTS OF THE INVENTION

The main object of the present invention is to provide a three-dimensional slow release agent absorbent dressing more suitable for use in sinus cavity or infective wounds; another object of the present invention is to provide a certain concentration of water-soluble agents mixed into dry absorbent articles. Said certain concentration is considered by the saturation capacity of said absorbent articles. After absorbent articles absorbs discharged exudate being in a saturated state, the certain concentration of dissolved agents will release and flow backward to the wound, which achieves the effect of producing bacteria static, lysis of necrotic tissue and even promoting granulation growth.

### BRIEF DESCRIPTION OF THE DRAWINGS

These and other features, aspects and advantages of the present invention will become apparent from the following description, claims and the accompanying embodiment shown in the drawing, which is briefly described below.

FIG. 1 is an enlarged cross-sectional view of the present invention of the three-dimensional slow release agent absorbent dressing.

FIG. 2 is a perspective view of the present embodiment of the three-dimensional slow release agent absorbent dressing.

### DESCRIPTION OF THE REFERRED EMBODIMENT

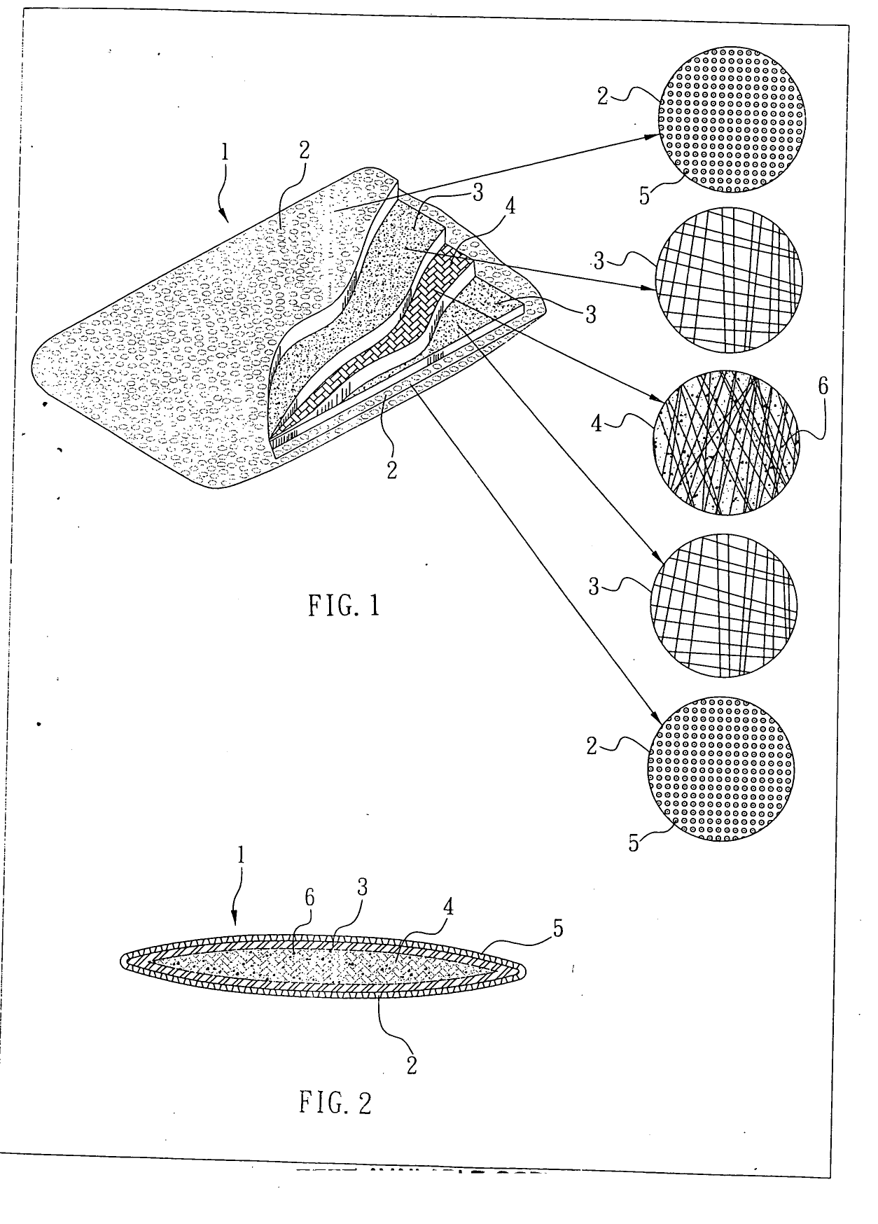

Referring to FIG. 1 and FIG. 2, the three-dimensional slow release agent absorbent dressing (1) of the present invention comprises a wound-contacting layer (2), a diffusion guiding layer (3) and absorbent articles (4). The placement of the above is one on top of another in order and the peripheral edges are joined together by heat-sealing. Wherein the wound-contacting layer (2), a first outside layer of the dressing (1), has a porous soft-film covered with tapered apertures (5). Said film can be a non-adherent polyethylene film and said tapered apertures (5) can be polygonal tapered or conical shaped apertures. The bottom surface of tapered apertures (5) with the largest caliber contacts a wound to produce a one-way valve and discharged exudate from the wound is able to penetrate through numerous tapered apertures (5) in many directions. And said discharged exudate is transmitted via the diffusion guiding layer (3) to absorbent articles (4) forming into the shape of gel to prevent from backflow. Said absorbent articles (4) are high-molecule absorbent materials, such as polyester or algin, mixed with the certain concentration of water-soluble agents (6). Said agents (6), can be antiseptic agents, enzymes and growth factor agents, in a suitable amount are added in dry absorbent articles (4) and said certain concentration is considered by the saturation capacity of said absorbent articles (4). After absorbent articles (4) absorbs discharged exudate being in a saturated state, the certain concentration of dissolved agents (6) will flow backward to the wound, which is more suitable for use in sinus cavity or infective wounds to achieve the effect of infection control. Therefore, as absorbent articles (4) are mixed with antiseptic agents, the dressing (1) has the function of producing bacteria static; as absorbent articles (4) are mixed with enzymes, the dressing (1) has the function of lysis of necrotic tissue and debridment and as absorbent articles (4) are mixed with growth factor agents, the dressing (1) has the function of promoting granulation growth. For example, the concentration of 1% Silver Nitrate (AgNO3) Solution is able to produce bacteria static. It assumes that the greatest amount of absorption capacity of 5 gm absorbent articles (4) in the dressing (1) is 50 cc. In the present embodiment, 0.5 gm Silver Nitrate is well mixed into 5 gm absorbent articles (4). After absorbent articles (4) absorb exudate in a saturated state, 1% Silver Nitrate (AgNO3) Solution will release from tapered apertures (5) of the wound-contacting layer (2) of the dressing (1) and flow backward to the wound bed acting. The wound-contacting layer (2) herein is formed of a soft-film covered with tapered apertures (5). Besides having the advantage of producing a one-way valve for discharging exudate, it is not adhering to the wound; for this reason, the dressing (1) is removed easily without hurting newborn epidermis. As shown in FIG. 1 and FIG. 2, the diffusion guiding layer (3) joined to the interior of the wound-contacting layer (2), can be directional fine-hole fibers or non-woven fabrics to transmit said discharged exudate to absorbent articles (4). Said absorbent articles (4) attached to the interior of said diffusion guiding layer (3) are high-molecule absorbent materials, such as polyester or algin. After absorbing discharged exudation, high-molecule polymeric fibers expand forming into the shape of gel to avoid exudate flowing backward to the wound. Said absorbent articles (4) are added with the certain concentration of said agents (6) in a suitable amount to absorb discharged exudate. Said agents (6) can be antiseptic agents, enzymes and growth factor agents. After being in a saturated state, the certain concentration of dissolved agents (6) will flow backward to the wound. In the present embodiment, non-woven pulp and high-molecule polymeric fibers under an antiseptic treatment are used.

What is claimed is:

1. A three-dimensional slow release agent absorbent dressing, comprising:
a wound-contacting layer, said wound-contacting layer being a first outside layer of said dressing and formed of a soft-film with tapered apertures;
a diffusion guiding layer, said diffusion guiding layer attached to an interior of said wound-contacting layer and being formed of directional fine-hole fibers or non-woven fabrics;
absorbent articles, said absorbent articles attached to an interior of said diffusion guiding layer, said absorbent articles being formed of high-molecule absorbent materials, said absorbent articles absorbing exudate discharged from a wound,
wherein said diffusion guiding disperses said exudate from said wound-contacting layer to said absorbent articles in a substantially uniform manner to increase absorbent capabilities of absorbent articles thereby preventing said exudate captured on said absorbent articles from passing back to said wound.

2. A three-dimensional slow release agent absorbent dressing, comprising:
a wound-contacting layer, said wound-contacting layer being a first outside layer of said dressing and formed of a porous soft-film with tapered apertures;
a diffusion guiding layer, said diffusion guiding layer attached to an interior of said wound-contacting layer and being formed of directional fine-hole fibers or non-woven fabrics;
absorbent articles, said absorbent articles attached to an interior of said diffusion guiding layer, said absorbent articles being formed of high-molecule absorbent articles and mixed with a certain concentration of water-soluble agents in a suitable amount, said absorbent articles absorbing exudate discharged from a wound,
wherein said diffusion guiding disperses said exudate from said wound-contacting layer to said absorbent articles in a substantially uniform manner to increase absorbent capabilities of absorbent articles thereby preventing said exudate captured on said absorbent articles from passing back to said wound.

3. A three-dimensional slow release agent absorbent dressing as recited in claim 2, wherein the certain concentration of water-soluble agents can be antiseptic agents, enzymes and growth factor agents and the concentration added to said absorbent articles in an amount based upon the saturation capacity of said absorbent articles, whereby once said-absorbent articles in a saturated state absorbs said exudate, said concentration dissolves and flows to said wound thereby promoting infection control, debridement and wound-healing.
